(19) United States
(12) Patent Application Publication
(10) Pub. No.: US 2021/0401399 A1
(43) Pub. Date: Dec. 30, 2021

(54) NANOPARTICLE-BASED IMAGING AND THERAPY

(71) Applicant: XENTER, INC., Salt Lake CIty, UT (US)

(72) Inventors: Richard J. Linder, Sandy, UT (US); David J. Bearss, Alipine, UT (US)

(21) Appl. No.: 17/357,525

(22) Filed: Jun. 24, 2021

Related U.S. Application Data
(60) Provisional application No. 63/044,963, filed on Jun. 26, 2020.

(51) Int. Cl. A61B 8/12; A61B 8/00; A61B 8/08; A61N 7/00; A61K 47/69

(52) U.S. Cl. CPC A61B 8/12; A61B 8/463; A61B 8/4494; A61B 8/0891; B82Y 5/00; A61K 47/6929; A61N 2007/0052; A61N 2007/0004; A61N 2007/0073; A61N 7/00

(57) ABSTRACT

The present disclosure relates generally to nanoparticle-based imaging, binding, and/or therapy at a targeted anatomical location within a subject. In particular, certain embodiments relate to intraluminal devices and systems configured to apply nanoparticles to an imaging target and/or treatment target within an anatomical lumen and to communicate imaging and/or treatment data wirelessly to one or more external devices.

### CROSS-REFERENCE TO RELATED APPLICATIONS

This application claims priority to and the benefit of U.S. Provisional Patent Application No. 63/044,963, filed Jun. 26, 2020 and titled “Nanoparticle-Based Imaging and Therapy”, the entirety of which is incorporated herein by this reference.

### BACKGROUND

Irregularities of various anatomical lumens, such as atherosclerosis and its accompanying vascular complications, remain a significant cause of patient morbidity and mortality, particularly in advanced and/or aging societies. Atherosclerosis is a condition where an artery wall thickens as a result of plaque accumulation. Atherosclerotic plaques are made up of lipids such as cholesterol, but additionally include many other types of substances and cells such as leukocytes, macrophages, neutrophils, eosinophils and other inflammatory cells, foam cells, cholesterol clefts, calcium, and fresh lipids. Over time, such plaques can lead to stenosis of the affected lumen. Other complications include rupture of the plaque (vulnerable plaques) and associated thrombotic embolism.

Detection and characterization of atherosclerotic plaques or other luminal irregularities (including lesions, clots, thrombi, and the like) remains challenging. Coronary angiography provides visualization of vessels and some organs of the body using an injected iodinated contrast agent and X-ray fluoroscopic imaging. Intravascular ultrasound (IVUS) emits sound waves from a catheter tip and detects return echo to provide an image of the vessel in the near vicinity of the catheter tip. Optical coherence tomography (OCT) is somewhat similar to IVUS but measures reflected infrared light rather than sound waves. Non-invasive imaging methods include variations of magnetic resonance imaging (MM) and computed X-ray tomography (CT).

While the foregoing methods have various strengths and weaknesses, the overall landscape of conventional techniques suffers from one or more of radiation exposure risk, the need for contrast agents, dyes, or other fluid infusions, sub-optimal imaging resolution, or inability to distinguish targets from surrounding tissues or structures.

The majority of conventional approaches to intraluminal imaging and/or treatment require the injection of dye and/or the use of X-rays. Each of these can be harmful to the subject. In addition, such imaging radiation can be harmful to the physicians and staff exposed to the radiation. Contrast radiopaque dye can damage the kidneys resulting in contrast induced nephropathy. Because of patient tolerance of radiopaque dyes and the risks of induced nephropathy, fluoroscopic evaluation and target vessel imaging is limited to ensure patient safety.

The use of catheters and other intraluminal devices can also be challenging due to the need to manage several long lengths of wires and other components, including guidewires, power cables, data wires, and the like. Care must be taken with respect to what is allowed in the sterile field and when it can be removed. Additional staff is often required simply to manage such wires and cables.

Accordingly, it would be advantageous to minimize the need for radiation and/or contrast agents in intraluminal imaging and/or treatment applications. There remains an ongoing need for systems, devices, and methods capable of improving upon conventional intraluminal imaging and/or treatment applications.

### SUMMARY

The present disclosure relates generally to nanoparticle-based imaging, binding, and/or therapy at a targeted anatomical location within a subject. In particular, certain embodiments relate to intraluminal devices and systems configured to apply nanoparticles to an imaging target and/or treatment target within an anatomical lumen and to communicate imaging and/or treatment data wirelessly to one or more external devices.

In one embodiment, an intraluminal system includes an intraluminal device (e.g., catheter or guidewire) and an external computer device communicatively coupled to the intraluminal device. The intraluminal device includes an imaging device associated with a distal portion of the intraluminal device. The imaging device is configured to image an intraluminal space and to generate nanoparticle-enhanced image data. The intraluminal device may additionally or alternatively include a treatment device associated with the distal portion of the intraluminal device. The treatment device is configured to apply energy to the surrounding lumen environment to cause nanoparticles within the lumen to increase in motion. The intraluminal device may also include one or more occluders (e.g., balloons) configured to limit passage of the nanoparticle composition beyond an area near the distal end of the intraluminal device.

In one embodiment, a method for imaging and/or treating an irregularity in an anatomical lumen comprises the steps of: advancing an intraluminal device within the anatomical lumen; delivering a nanoparticle composition to the anatomical lumen (e.g., by way of a lumen of the intraluminal device) and allowing the nanoparticle composition to preferentially interact with an irregularity of the lumen; and performing one or both of (a) activating the imaging device to image the lumen with images enhanced by the nanoparticles; (b) activating the treatment device to cause the nanoparticles to increase in motion and thereby treat the irregularity.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosed embodiments will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A and 1B illustrate placement of nanoparticle-probe conjugates in a target lumen (a blood vessel in this example) and association of the conjugates with a plaque disposed within the lumen;

FIG. 2 illustrates an exemplary intraluminal system including an intraluminal device in the form of a catheter and including an external device communicatively coupled to the catheter to receive data from the catheter;

FIG. 3A illustrates an exemplary intraluminal system including an intraluminal device in the form of a guidewire and including an external device communicatively coupled to the guidewire to receive data from the guidewire;

FIG. 3B illustrates another example of an intraluminal device in the form of a stent delivery device;

FIG. 3C illustrates another example of an intraluminal device in the form of a balloon device;

FIG. 4 illustrates positioning of the distal end of an intraluminal device within a vessel for imaging of a plaque within the vessel;

FIG. 5 illustrates positioning of the distal end of an intraluminal device within a vessel for treatment of a plaque within the vessel; and

FIG. 6 illustrates positioning of the distal end of an intraluminal device within a vessel and using one or more balloons or other occlusive devices to limit passage of nanoparticles beyond a targeted region of the vessel.

### DETAILED DESCRIPTION

### Nanoparticles & Nanoparticle-Probe Conjugates

Nanoparticles utilized in the disclosed embodiments may comprise a metal, oxide, polymer, or combination thereof. In some embodiments, metal nanoparticles can comprise one or more of gold, platinum, silver, palladium, rhodium, osmium, ruthenium, rhenium, molybdenum, copper, iron, nickel, tin, beryllium, cobalt, antimony, chromium, manganese, zirconium, zinc, tungsten, titanium, vanadium, lanthanum, cerium, heterogeneous mixtures thereof, alloys thereof, or combinations thereof. Gold nanoparticles have low toxicity and several other benefits for biological applications, but other metals and/or other materials may be utilized according to particular application needs. Perfluorocarbon nanoparticles may be particularly suitable for imaging applications and may be utilized alone or in combination with one or more other nanoparticle types disclosed herein.

Nanoparticles are often defined as any particle having at least one dimension measuring less than 100 nm. Nanoparticles may have an average size of about 10 nm to 100 nm. Although nanoparticles in accordance with such sizes are preferred, other particles may also be utilized in some embodiments. For example, larger particles that may be considered microparticles may alternatively be utilized in at least some circumstances.

To enable interaction with an imaging and/or treatment target, the nanoparticles may be functionalized. For example, the nanoparticles may be conjugated with one or more probe molecules to form particle-probe conjugates. Probe molecules may comprise peptides, proteins, antibodies, nucleic acids, glycoproteins, or other biomolecules. Nanoparticles, particularly metal nanoparticles, typically have effective bonding with thiol, disulfide, and/or amine groups, so probe molecules may include a peptide or protein linker that attaches to the nanoparticle at one section and attaches to another biomolecule at another section. The other biomolecule may be another peptide or protein, or may be a different type of biomolecule, such as a lipid, carbohydrate, and/or nucleic acid. Probe molecules may have particular functionality and may comprise an antibody, antigen, and/or other ligand. Probe molecules may comprise a fluorescent label. Other examples of molecules or compounds that may be utilized to functionalize the nanoparticles include: proteases and other enzymes such as thrombin, tissue plasminogen activator (TPA), streptokinase (SK), and urokinase (uPA); anticoagulant medications such as heparin and/or other glycosaminoglycans; and molecules associated with coagulation or anticoagulation pathways, or related variants, such as protein S (PROS) and GAS 6.

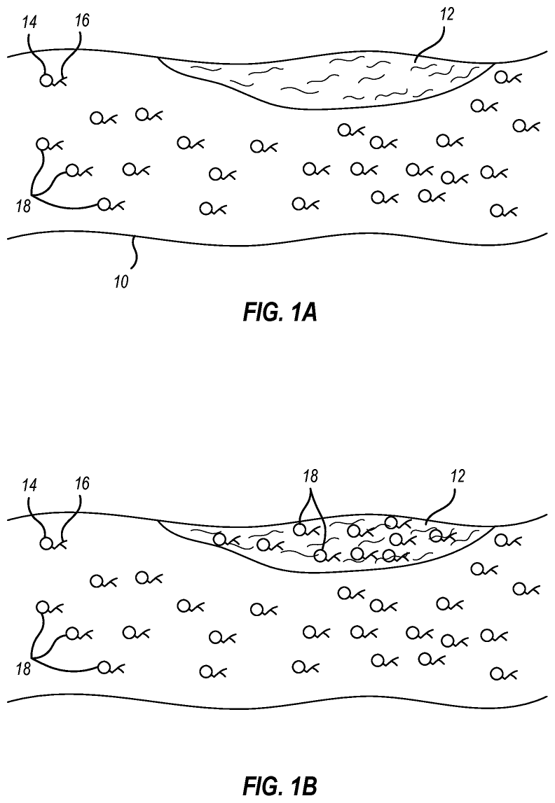

The one or more probe molecules can be selected to enable the particle-probe conjugates to preferentially interact with an imaging and/or treatment target. FIG. 1A schematically illustrates the introduction of a plurality of particle-probe conjugates 18 into a biological lumen 10. The particle-probe conjugates 18 comprise nanoparticles 14 modified by one or more probe molecules 16. The lumen 10 includes an irregularity such as a lesion, plaque, clot, thrombus, and/or other vascular or luminal irregularity. FIG. 1A can represent, for example, a vessel 10 in which an atherosclerotic plaque 12 is present. The plaque 12 is a complex mixture of different lipids, cell types (including various leukocytes and epithelial cells), and other biomolecules. The composition of the plaque 12 includes certain cells and/or molecules that are present in higher concentration relative to the surrounding tissues of the vessel wall. With appropriate probe selection, this allows the particle-probe conjugates to preferentially bind and/or interact with the surface and/or internal structures of the plaque 12, as shown in FIG. 1B.

The particle-probe conjugates 18 may be administered systemically. Alternatively, and more preferably, the particle-probe conjugates 18 may be delivered in a targeted, localized manner to limit distribution of the particle-probe conjugates 18 and/or limit the total amount of particle-probe conjugates 18 required to achieve desired imaging and/or treatment outcomes. Features for achieving such localized delivery are described in more detail below.

Imaging targets may vary depending on particular anatomical conditions such as the type of lumen targeted, the type of plaque/irregularity being treated, and the desired sections or metabolic processes targeted for preferential attachment of the particle-probe conjugates. In vascular plaques, for example, imaging targets may include one or more of VCAM-1, ICAM-1, E-selectin, P-selectin, FDG, FCH, HDL, LDL, CD68, LOX-1, SRs, MPO, phosphatidylserine, MMPs, cathepsins, collagen, αvβ3 integrin, hydroxyapatite, glycoprotein IIb/IIa, fibrin, and/or factor XIII See, for example, Quillard and Libby “Molecular Imaging of Atherosclerosis for Improving Diagnostic and Therapeutic Development” Circulation Research, Vol. 111, No. 2.

In some embodiments, a nanoparticle composition includes nanoparticles and/or nanoparticle-probe conjugates and a carrier. The carrier may include a medically appropriate aqueous solution, such as a saline solution. Other embodiments may include a carrier in the form of a gel or paste. The nanoparticles and/or nanoparticle-probe conjugates can thus be mixed with the carrier to form a colloidal solution or an emulsion, depending on carrier properties.

### Overview of Intraluminal Systems

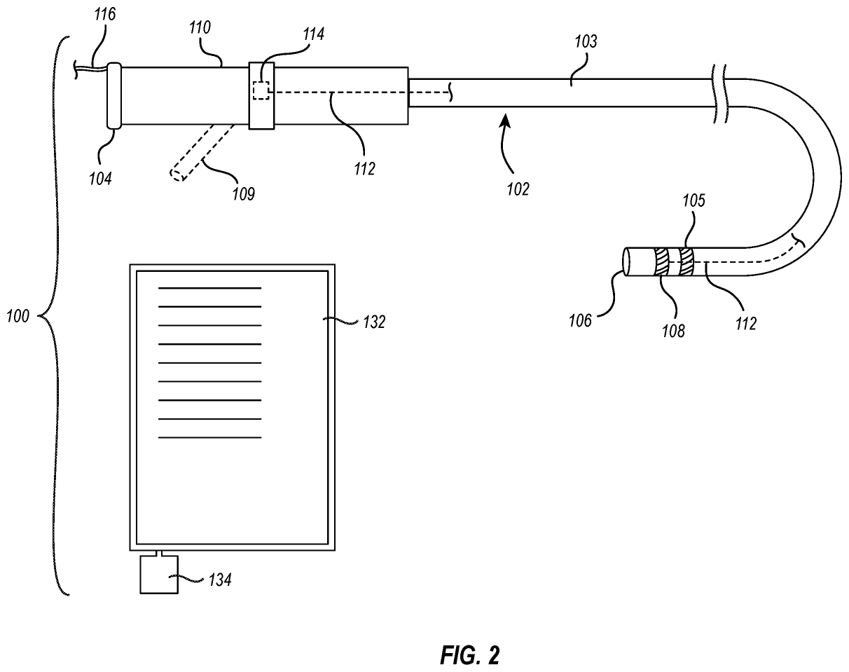

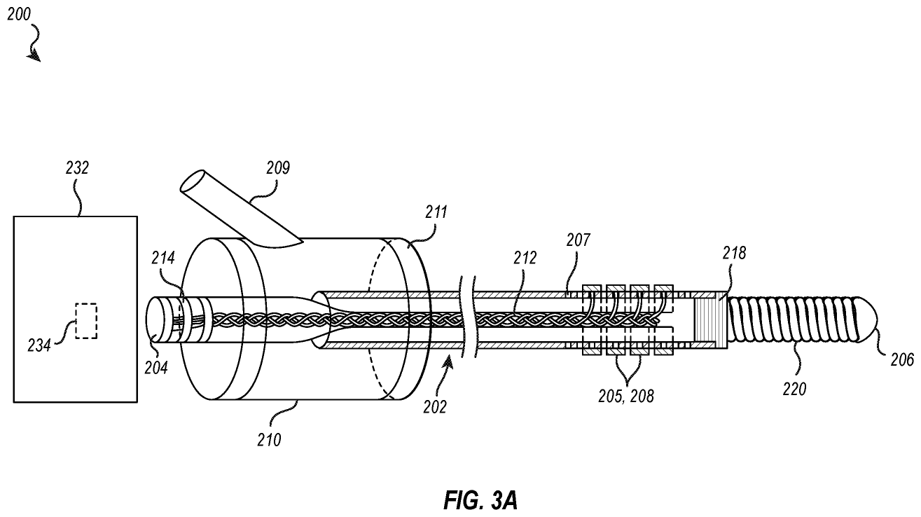

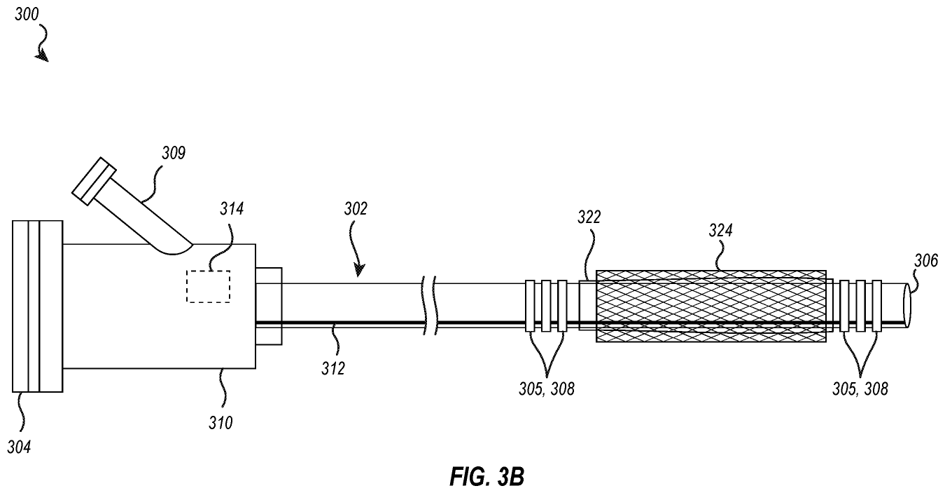

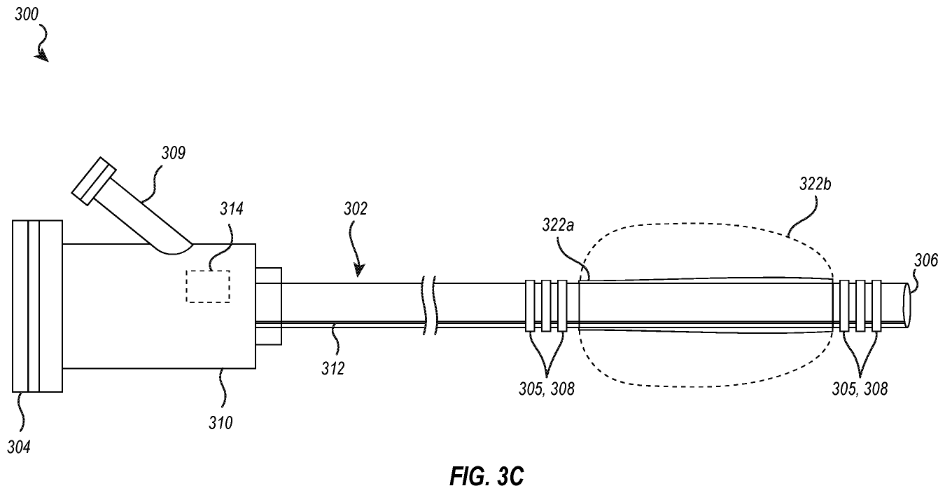

FIGS. 2, 3A, and 3B illustrate exemplary intraluminal systems 100, 200, and 300, respectively, that may be utilized to provide one or more of delivery of particle-probe conjugates 18 to a targeted lumen, imaging of the target lumen, or treatment of the target lumen. FIG. 2 illustrates an intraluminal system in the form of a catheter system, FIG. 3A illustrates a guidewire system, and FIG. 3B illustrates a stent delivery system. The skilled person will recognize that the principles described herein may be applied using any of the illustrated forms of intraluminal devices, or with other forms of intraluminal devices. For catheter-based embodiments, the principles and features described herein are applicable to catheter or microcatheter embodiments.

Referring to FIG. 2, a catheter system 100 includes a catheter 102 with a length extending from a proximal end 104 to a distal end 106. The catheter 102 includes one or more imaging devices 108 disposed at a distal portion. In use, an imaging device 108 is configured to gather nanoparticle-enhanced image data within the lumen in which it is positioned. The catheter 102 may additionally or alternatively include one or more treatment devices 105 disposed at a distal portion. In use, a treatment device 105 is configured to provide a “trigger” to nanoparticles (e.g., by providing energy to the nanoparticles) within the vicinity of the treatment device 105 that enables the nanoparticles to further interact with or treat the tissues the nanoparticles are associated with.

In some embodiments, as described in more detail below, a single component functions as both a treatment device 105 and an imaging device 108. For example, some embodiments include an ultrasound transducer (or an array of ultrasound transducers) capable of providing ultrasound-based imaging (and therefore functioning as an imaging device 108) as well as providing ultrasonic energy for activating nanoparticles (and therefore functioning as a treatment device 105). In some embodiments, wherein an ultrasound transducer (or array of ultrasound transducers) is used as both the imaging device and the treatment device, the ultrasound transducer(s) may operate at a first frequency while functioning as the imaging device and operate at a second frequency when functioning as the treatment device, the second frequency being different than the first frequency and depending, at least in part, on the energy required to activate the nanoparticles. Imaging devices disclosed herein, including those that incorporate one or more ultrasound transducers, may be configured for forward-looking applications and/or side-looking applications. Embodiments that incorporate one or more of such bi-functional ultrasound transducers may be provided according to any of the intraluminal systems and devices described herein, including catheter, microcatheter, guidewire, and delivery device (e.g., stent delivery device) embodiments.

For embodiments that incorporate one or more ultrasound transducers, the frequencies utilized for imaging and the frequencies utilized for nanoparticle activation may be the same or different based on particular nanoparticle configurations and/or application needs. Typically, an ultrasound transducer operates with a center frequency of about 5 to about 50 MHz, or about 8 to about 40 MHz, or about 10 to about 30 MHz, or other ranges using any two of the foregoing values as endpoints. Frequencies for activating nanoparticles will most often depend on resonant properties of the nanoparticles themselves. The skilled person, in light of this disclosure, is able to determine effective frequencies for activating a given type and size of nanoparticle.

Although most of the following examples will refer to positioning of the catheter 102 within a blood vessel, it will be understood that the disclosed embodiments are not necessarily limited to vascular applications and may alternatively be utilized in other applications involving other anatomical lumens, such as applications involving the gastrointestinal tract, pathways of the bronchi, renal tubules, urinary ducts, or genital tracts, for example. Further, although many of the examples described herein will refer specifically to the human body, it will be understood that the same embodiments and principles may be utilized in applications involving other animals, and in particular other mammals.

The catheter 102 is configured to relay sensed data pertaining to the imaging and/or treatment to one or more external devices 132. The one or more external devices 132 may assist the physician in viewing and analyzing the targeted lumen and plaques or other irregularities disposed therein, allowing the physician to better understand the environment of the lumen in the vicinity of the catheter tip so that the physician can make appropriate decisions while treating a patient.

The catheter 102 may be sized and configured to be temporarily inserted in the body, or implanted in the body, or configured to deliver an implant in the body. In one embodiment, the catheter 102 may be of the type to act as a peripherally inserted central catheter (PICC) line, typically placed in the arm or leg of the body to access the vascular system of the human body. The catheter 102 may also be a central venous catheter, an IV catheter, or any other type of catheter sized and configured to be positioned within a lumen of the body, such as a coronary catheter, stent delivery catheter, balloon catheter, atherectomy type catheter, and/or an imaging catheter. The catheter 102 may be primarily formed of polymeric materials with metallic materials embedded therein.

Intraluminal devices described herein include one or more lumens extending along a length of the device through which a nanoparticle composition may be delivered. The illustrated catheter 102, for example, includes a single lumen 103, though other embodiments include multiple lumens. For example, a multi-lumen catheter may be configured such that one or more lumens are configured for delivering nanoparticles or nanoparticle-probe conjugates and one or more different lumens are configured for delivering other devices, fluids, medications, etcetera. Other lumens or ports may extend transverse relative to the longitudinal axis of the catheter 102 or may extend substantially parallel with the lumen 103.

The catheter 102 may include a hub or housing 110 at or near the proximal end 104. The housing 110 may be configured to facilitate longitudinal translation of the catheter tube relative to the housing 110 such that the catheter 102 may be lengthened or shortened (e.g., extended into or retracted from within a body), depending upon the needs of the physician. The housing 110 may include one or more seals, such as one or more O-ring seals to engage corresponding external surface(s) of the catheter tube while also substantially preventing fluid from leaking from the catheter 102 and housing 110. The housing 110 may further include one or more connectors and/or one or more additional ports 109 to facilitate delivery of fluids through the catheter 102.

The one or more imaging devices 108 and the one or more treatment devices 105 may be integrally formed in the tip portion (i.e., the distal portion) of the catheter 102 by employing bonding, molding, co-extrusion, welding, gluing, and/or other techniques or manufacturing processes. As shown, one or more power and/or data wires 112 extend from the imaging device 108 and treatment device 105 at the distal portion of the catheter 102 to the proximal sections of the catheter 102. The wires 112 may extend to a transmitter 114 and/or a power supply 116. The power supply 116 may include a wire that extends from the housing 110 for connection to an appropriate power source. Alternatively, the catheter 102 may be configured to run on battery power. One or more batteries may be placed in the housing 110, for example.

The transmitter 114 may be positioned adjacent the proximal portion of the catheter 102, such as in the housing 110. Additionally, or alternatively, the transmitter 114 may be provided in a separate assembly connected or disconnected from the housing 110. Additionally, or alternatively, a transmitter 114 may be positioned adjacent the one or more imaging devices 108 or treatment devices 105. In some embodiments, for example, a transmitter 114 may be embedded within the wall of the catheter 102.

The transmitter 114 operates to wirelessly relay or transmit data received from the imaging device(s) 108 and/or treatment device(s) 105 to a receiver 134 associated with an external device 132. Some embodiments may include or allow for a wired connection between the catheter 102 and the external device 132 in order to enable data transfer, though configuring the system 100 for wireless data transmission is preferred in order to avoid the need for additional wire management.

The one or more imaging devices 108 and one or more treatment devices 105 are preferably sized to allow effective placement at the distal portion of an intraluminal device. The sizes of these devices have practical limits based on the intended application of the intraluminal device. For example, for most intravascular applications, the devices have a size limit (e.g., in the largest cross-sectional dimension) of about 0.300 inches, and are more preferably equal or less than about 0.200 inches or even more preferably equal or less than about 0.100 inches. In other applications, however, larger sizes may be suitable due to the larger luminal spaces involved. For example, other applications may allow for sizes up to 0.375 inches, or up to about 0.475 inches, or even up to about 1-2 inches.

The one or more imaging devices 108 may relay imaging data, such as pixel arrays, images, video, or other types of imaging data, via the relay wires 112 to the transmitter 114, which may then transmit data to the receiver 134 and may be for storage, display, and/or processing at the external device 132. The imaging device 108 may comprise any imaging modality known in the art suitable for positioning at or integration with a distal portion of an intraluminal device, including a fiber-optic camera, LIDAR system, Raman scattering system, mm wave camera, infrared imaging system, radiofrequency imaging system, other imaging devices/systems known in the art, or combinations thereof. Image data gathered by the imaging device 108 may be modified using one or more image enhancing algorithms known in the art.

The one or more treatment devices 105 are primarily configured to emit energy in a controlled manner in order to activate nanoparticles or the particle-probe conjugates in the vicinity of the treatment device 105. The energy supplied by the treatment device 105 may be in the form of an electric field, a magnetic field, light, mechanical energy (e.g., ultrasound) or heat, for example. In some embodiments the treatment device 105 can emit a chemical signal intended to activate the nanoparticles or the particle-probe conjugates. The applied energy can modulate the nanoparticles or the particle-probe conjugates to provide desired treatment effects. For example, the applied energy can cause the nanoparticles or the particle-probe conjugates to oscillate, rotate, or otherwise move in response. In some applications, this can promote the disruption of bonds between cells and other connected molecules of the treatment target. Where the treatment target is a plaque, for example, the applied energy from the treatment device 105 can cause the nanoparticles or the particle-probe conjugates to promote dislodging of the plaque and breakup of the plaque into smaller, less dangerous pieces. In some embodiments, the applied energy may result in a cracking of a calcified lesion, for example, to enable optimal stent expansion.

The guidewire system 200 illustrated in FIG. 3A includes features similar to those of the catheter system 100 of FIG. 2, and the foregoing description is applicable to the guidewire system 200, with a few additional or alternative features noted below. The guidewire system 200 includes a guidewire 202 extending from a proximal end 204 to a distal end 206, and including one or more imaging devices 208 and/or treatment devices 205. In this embodiment, the guidewire core 212 itself may function as the structure that relays power and/or data between one or more distal sensors (e.g., the imaging devices 208 and/or treatment devices 205) and a proximal device such as housing 210. Additional examples and details related to embodiments that utilize the guidewire core itself to pass signals between one or more distal sensors and the housing 210 or other proximal device are provided in U.S. patent application Ser. No. 17/205,964, which is incorporated herein in its entirety.

In some embodiments, the intraluminal device is configured to pass power and/or data signals between one or more distal sensors (e.g., imaging devices 208 and/or treatment devices 205) and a proximal device by allocating a signal space into a plurality of unique contiguous regions of frequency, and uniquely allocating each of the plurality of unique contiguous regions of frequency to (i) one or more power channels or (ii) one or more sensor signal channels (i.e., channels for signals from the imaging devices 208 and/or treatment devices 205). Additional examples and details related to embodiments that utilize frequency channels for passing signals on the device are provided in U.S. patent application Ser. No. 17/205,614, which is incorporated herein in its entirety. Such frequency channel allocation can also be utilized in other types of intraluminal systems described herein (e.g., catheter system 100 and stent delivery system 300).

In the illustrated embodiment, a transmitter 214 is electrically connected to the imaging devices 208 and/or treatment devices 205 and the transmitter 214 is communicatively coupled to a receiver 234 associated with an external device 232 to enable wireless (or optionally, wired) transmission of imaging and/or treatment data from the guidewire 202 to the external device 232. In some embodiments, the transmitter 214 is incorporated into the housing 210. In some embodiments, the housing 210 is configured as a power and data coupling device configured to capacitively couple to an elongated conductive member (e.g., the core 212) of the intraluminal system 200 such that no direct physical contact is required between the elongated conductive member and the housing 210. Additional examples and details related to embodiments that utilize a power and data coupling device are provided in U.S. patent application Ser. No. 17/205,754, which is incorporated herein in its entirety. Such power and data coupling devices can also be utilized in other types of intraluminal systems described herein (e.g., housing 110 of catheter system 100 and housing 310 of stent delivery system 300).

The housing 210 may also include one or more ports 209 and/or one or more seals 211 as part of a hemostasis valve. The guidewire 202 may also include distal ports 207 to allow delivery of fluids to areas adjacent distal regions of the guidewire 202. The distal ports 207 may be located coincident with or near the imaging device(s) 208 and/or treatment device(s) 205. The guidewire 200 may also include a core tip 218 and coil 220 surrounding the core tip 218 near the distal end of the guidewire 200. The distal end 206 can include a rounded or ball-shaped, atraumatic shape. Other guidewire structures known in the art may also be additionally or alternatively utilized.

FIG. 3B illustrates an example of an intraluminal device in the form of a stent-delivery system 300. The stent-delivery system 300 includes features similar to those of the catheter system 100 and guidewire system 200, and the foregoing description for these other types of intraluminal systems is also applicable to the stent-delivery system 300, with a few additional or alternative features noted below. Similar embodiments may be configured for delivering other interventional and/or implantable devices.

The stent-delivery system 300 includes a housing 310 at or near its proximal end 304. The housing 310 may include one or more additional ports 309. A delivery catheter 302 extends from the housing 310 to a distal end 306. The stent-delivery system 300 also includes a balloon 322 and associated stent 324. As known in the art, the delivery catheter 302 may be routed through the anatomy such that the pre-deployed stent 324 is positioned at a target location. The balloon 322 is then inflated (e.g., by passing fluid and/or gas through the catheter 302 and through ports associated with the balloon 322) to thereby expand the stent 324 at the target location.

As shown, the illustrated stent-delivery system 300 also includes one or more imaging devices 308 and/or one or more treatment devices 305. These may be positioned proximal of the balloon 322 and stent 324, distal of the balloon 322 and stent 324, or both proximal and distal of the balloon 322 and stent 324. Additionally, or alternatively, one or more imaging devices 308 and/or one or more treatment devices 305 may be coincident with the balloon 322 and/or stent 324.

In another embodiment, a balloon system may be used without a stent. For example, a balloon system (i.e., balloon device) may be substantially similar to the stent-delivery system 300 except not include the stent itself. Such a system is illustrated in FIG. 3C, with reference numbers corresponding to the reference numbers of the system of FIG. 3B, but without a stent 324. In such a balloon system, the balloon 322 (shown as 322a in a deflated configuration and 322b in an inflated configuration) is inflated to place pressure against the walls of a vessel (e.g., at the location of a lesion) while a treatment device is operated. For example, prior to the inflation of the balloon 322, functionalized nanoparticles or particle-probe conjugates may be introduced to preferentially bind or otherwise interact with the lesion. The balloon 322 may then be inflated and a treatment device 305 may provide appropriate energy to activate the nanoparticles while the balloon 322 simultaneously applies pressure to the vessel walls at the site of the lesion. In some embodiments, the treatment device may pass energy (e.g., ultrasound waves) through the fluid medium contained within the balloon 322 (e.g., saline) when it is inflated and applying pressure to the vessel wall. Thus, the balloon 322 and the energy-activated nanoparticles may act in concert to crack a calcified region or otherwise treat such an irregularity. As with other embodiments, and as has been previously described, imaging may occur prior to such treatment using the balloon system.

In another embodiment using a balloon device, the balloon 322 may be coated with modified nanoparticles (e.g., drug coated, conjugated, or otherwise functionalized). Expansion of the balloon (e.g., from the deflated configuration 322a to the inflated configuration 322b) may cause the modified nanoparticles to be released at the site of the irregularity. Thus, the balloon 322 may be a delivery mechanism, a treatment mechanism (through inflation and application of pressure to the treatment site, through delivery of energy to the nanoparticles at the treatment site, or both), or it may function in both capacities.

As with other embodiments, the use of balloon device may be carried out using a catheter, a microcatheter or a guidewire. In some embodiments, combinations of such devices may be used. For example, a catheter may be used to deliver the nanoparticles and to position and inflate a balloon. Such a catheter may be a multi-lumen catheter with one lumen delivering nanoparticles and another lumen delivering a fluid for inflation of the balloon. A guidewire having a treatment mechanism (e.g., ultrasound transducers) may be used to activate the nanoparticles.

In some embodiments, methods of treatment may include delivering the nanoparticles to a treatment site, treating the irregularity by activating the nanoparticles, and then returning to the irregularity at some later time to again activate any nanoparticles remaining at the site of the irregularity. In some cases, the follow-up treatment (e.g., the return to the irregularity with a treatment device and the re-activation of the nanoparticles) may be minutes, hours, days, weeks, or even months subsequent to the initial treatment or activation of the nanoparticles.

As with the other intraluminal systems described herein, the stent-delivery system 300 (as well as a similarly configured balloon system) includes a transmitter 314 configured to receive signals from the one or more imaging devices 308 and/or treatment devices 305. The transmitter 314 may be communicatively coupled to a receiver associated with an external device to enable wireless (or optionally, wired) transmission of imaging and/or treatment data from the stent-delivery device 300 to the external device. In the illustrated embodiment, the transmitter 314 is integrated with or otherwise associated with the housing 310, though alternative embodiments may dispose the transmitter 314 elsewhere, such as at a proximal portion of the catheter 302.

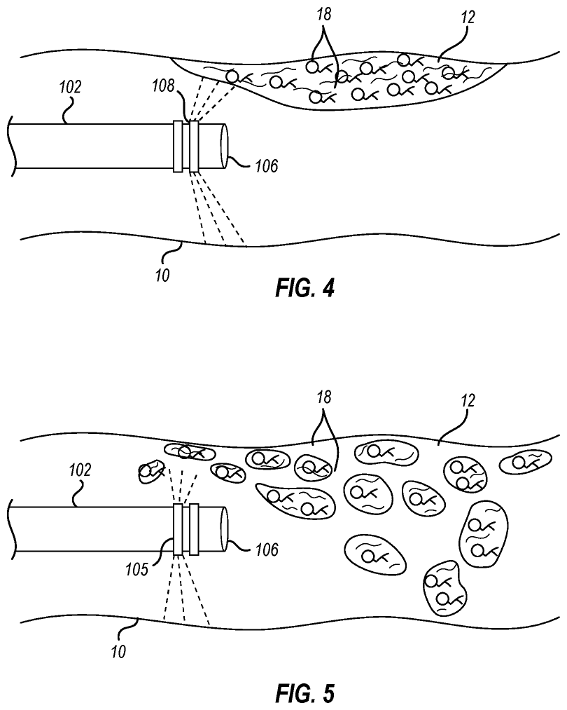

FIG. 4 illustrates an exemplary use of the catheter 102 to provide imaging of the target lumen 10, including imaging of an irregularity such as plaque 12 within the lumen. Similar methods may be carried out using other types of intraluminal systems described herein, such as guidewire system 200 and stent-delivery device 300. As shown, the plaque 12 has bonded or otherwise interacted with a concentrated dose of particle-probe conjugates 18. Operation of the imaging device 108 allows for the measure of the radial distance from the imaging device 108 to the walls of the lumen 10 and to the edge of the plaque 12. The concentrated presence of the nanoparticles on the surface of the plaque 12 and/or within the plaque 12 enables enhanced imaging of the plaque 12. That is, the nanoparticles can provide greater reflectance and a greater corresponding imaging signal, for example.

FIG. 5 illustrates an exemplary use of the catheter 102 to provide treatment of the target lumen 10 by treating a plaque 12 disposed therein. Similar methods may be carried out using other types of intraluminal systems described herein, such as guidewire system 200 and stent-delivery system 300. As shown, the treatment device 105 is activated and the applied energy causes the nanoparticles or particle-probe conjugates to respond by oscillating, rotating, otherwise increasing in movement, changing in temperature, changing phase, or otherwise exhibiting a material or chemical change. This can beneficially disrupt the connective bonds holding the plaque 12 together, causing it to crack, detach from the vessel wall and/or break up into smaller pieces 13. The smaller pieces 13 are preferably small enough to be more safely managed by the body as compared to a larger, riskier embolus.

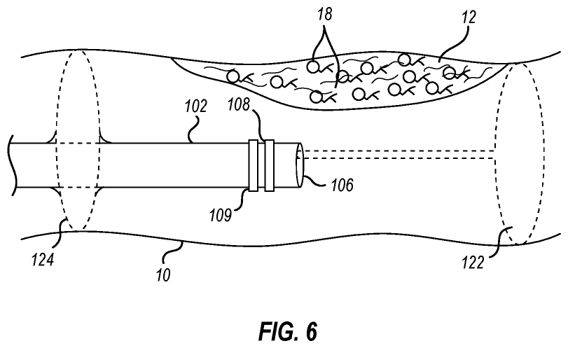

FIG. 6 illustrates an application that allows the particle-probe conjugates 18 to be locally applied rather than systemically administered. This example illustrates an exemplary process using catheter 102 of catheter system 100, but the same features and steps may be carried out using other intraluminal systems described herein, such as guidewire system 200 and stent-delivery system 300. One or more occluders may be positioned within the lumen 10 to limit passage of the particle-probe conjugates 18 beyond the occluder(s). The occluders may be balloons, or other selectively expandable and retractable occlusive devices known in the art. A distal occluder 122 may be positioned distal of the distal end 106 of the catheter 102 to limit distal passage of the particle-probe conjugates 18. Additionally, or alternatively, a proximal occluder 124 may be positioned proximal of the distal end 106 of the catheter 102 to limit proximal passage of the particle-probe conjugates 18. Whether one or both of occluders 122 and 124 are deployed may depend on the target lumen, the direction of fluid (e.g., blood) flow, and whether the catheter 120 is inserted in a retrograde or antegrade direction relative to blood flow, for example. Imaging and/or treatment (including the targeted deployment of nanoparticles or particle-probe conjugates) may then be carried out within the local space of the target anatomy.

In some embodiments, similar to some of the features and concepts set forth herein, nanoparticles or the particle-probe conjugates may be associated with (e.g., serve as a coating on or otherwise be integrated with) an implantable device such as a stent, cardiac implant, blood filter, or other device delivered intraluminally before being detached from a delivery system and left within the target lumen of the body.

### Communication to External Devices

Referring again to FIG. 2 (with the understanding that the same description is applicable to corresponding features of other intraluminal systems such as illustrated in FIGS. 3A and 3B as well), the external device 132 may be a hand-held device, such as a smart phone, tablet, lap-top computer, or any other external device with a processor, memory, an input portion, output portion, and a power source. The power source may be a rechargeable battery, for example. The input portion may include a touch sensitive screen, microphone, keyboard, mouse, and/or input buttons. The output portion may include a viewable display, speakers, and the like. The processor and memory may process and hold the data, which may be formatted and viewable on the display with software held in the memory, as known to one of ordinary skill in the art. Such software may be downloadable to the external device 132 as application software to be readily employed by physicians in conjunction with the catheter system 100.

Although exemplary embodiments are described herein as using hand-held or mobile devices as the one or more external devices 132, it will be understood that this is not necessary, and other embodiments may include other “non-mobile” computer systems that may include a desktop computer, monitor, projector, and the like. In some embodiments, the one or more external devices 132 includes a mobile/hand-held device and additionally includes a desktop device or other non-mobile device. For example, the mobile device may be configured to receive transmitted data from the transmitter 114 of the catheter 102 and function as a bridge by further sending the data to the non-mobile computer system. This may be useful in a situation where the physician would like the option of viewing data on a mobile device but may need to have the data additionally or alternatively passed or mirrored on a larger monitor such as when both hands are preoccupied (e.g., while handling the catheter).

The receiver 134 that may be plugged into an input port of the external device 132, with the receiver 134 sized and configured to receive data transmitted from the transmitter 114 of the catheter 102 via one or more acceptable wireless systems, as known by one of ordinary skill in the art. The wireless system(s) may include, for example, a personal area network (PAN) (e.g., ultra-high frequency radio wave communication such as Bluetooth®, ZigBee®, BLE, NFC), a local area network (LAN) (e.g., WiFi), or a wide area network (WAN) (e.g., cellular network such as 3G, LTE, 5G). In some embodiments, the receiver 134 is internal to the device 132 and the device 132 does not necessarily need to include an external receiver 134 that is plugged into a port of the device 132. Wireless data transmission may additionally or alternatively include the use of light signals (infrared, visible radio, with or without the use of fiber optic lines), such as radiofrequency (RF) sensors, infrared signaling, or other means of wireless data transmission.

The external device may operate to format the signals received from the catheter 102 to provide characterization data related to the anatomical target lumen (e.g., vessel) and/or environment adjacent the tip portion of the catheter 102. The processing may be fully or primarily carried out at the external device 132, or alternatively may be at least partially carried out at one or more other external devices communicatively connected to the external device 132, such as at a remote server or distributed network. Such processing may operate to format the data into a useful form that characterizes various parameters and the environment within the target anatomy to assist the physician for appropriately treating the patient. Such characterization data may be saved in the memory of the external device 132 and/or in memory of one or more other external devices or networks.

In another embodiment, additional, remote external devices may be coupled, linked, or associated with the catheter system 100 such that an authorized physician or other person at a remote location can review, analyze and/or monitor (in real-time or reviewed/analyzed later) the characterization data to, for example, assist in making decisions for a patient. Such remote location may be within the hospital or clinic where the patient is being treated or may be outside (remote of) the hospital or clinic.

### Additional Exemplary Aspects

Embodiments of the present disclosure may include, but are not necessarily limited to, features recited in the following clauses:

Clause 1: An intraluminal system configured to be at least partially positioned within an anatomical lumen of a subject, the intraluminal system comprising: an intraluminal device having a length extending between a proximal end and a distal end, the intraluminal device defining a lumen along the length thereof throughwhich a nanoparticle composition may be delivered, the intraluminal device including a distal portion with an imaging device associated therewith, the imaging device configured to image an intraluminal space and to generate nanoparticle-enhanced image data; and optionally an external device operatively coupled to the imaging device, the external device configured to receive the image data from the imaging device.

Clause 2: The intraluminal system of Clause 1, further comprising a transmitter and a receiver, the transmitter coupled to the intraluminal device and the receiver coupled to the external device.

Clause 3: The intraluminal system of Clause 2, further comprising a housing associated with a proximal portion of the intraluminal device, the transmitter being integrated with the housing.

Clause 4: The intraluminal system of Clause 3, further comprising one or more power and/or data wires connecting the imaging device at the distal portion of the intraluminal device to the transmitter at the proximal portion of the intraluminal device.

Clause 5: The intraluminal system of any one of Clauses 1-4, wherein the external device operates to display the image data received from the imaging device on a display screen of the external device to thereby display nanoparticle-enhanced images of the intraluminal space.

Clause 6: The intraluminal system of any one of Clauses 1-5, wherein the lumen of the intraluminal device includes a lumen wall defining the lumen of the intraluminal device, the imaging device being integrated with the lumen wall.

Clause 7: The intraluminal system of any one of Clauses 1-6, wherein the imaging device comprises an ultrasound transducer.

Clause 8: The intraluminal system of Clause 7, wherein the ultrasound transducer is configured as a forward-looking ultrasound transducer.

Clause 9: The intraluminal system of Clause 7, wherein the ultrasound transducer is configured as a side-looking ultrasound transducer.

Clause 10: The intraluminal system of any one of Clauses 1-9, further comprising a treatment device associated with the distal portion of the intraluminal device, wherein the treatment device is configured to apply energy to the intraluminal space to activate nanoparticles disposed therein.

Clause 11: The intraluminal system of Clause 10, further comprising an ultrasound transducer or an array of ultrasound transducers configured to function as both the imaging device and the treatment device.

Clause 12: The intraluminal system of claim 11, wherein the ultrasound transducer or the array of ultrasound transducers operate at a first frequency when functioning as the imaging device and operate at a second, different frequency when functioning as the treatment device.

Clause 13: The intraluminal system of any one of Clauses 1-12, further comprising one or more occluders configured to limit passage of the nanoparticle composition beyond an area near the distal end of the intraluminal device.

Clause 14: The intraluminal system of any one of Clauses 1-13, wherein the intraluminal device is a catheter.

Clause 15: The intraluminal system of any one of Clauses 1-13, wherein the intraluminal device is a guidewire.

Clause 16: The intraluminal system of any one of Clauses 1-13, wherein the intraluminal device is a balloon device.

Clause 17: The intraluminal system of any one of Clauses 1-13 or Clause 16, wherein the intraluminal device is a stent-delivery device.

Clause 18: A kit for imaging and/or treating an irregularity in an anatomical lumen, the kit comprising: an intraluminal device (such as in any one of Clauses 1-17) that includes a proximal end, a distal end, and a lumen extending therebetween through which a nanoparticle composition may be delivered, and a distal portion with an imaging device, a treatment device, or both associated therewith; and a nanoparticle composition for passing through the lumen of the intraluminal device, the nanoparticle composition comprising nanoparticle-probe conjugates, the nanoparticle-probe conjugates comprising one or more probe molecules coupled to the nanoparticles.

Clause 19: The kit of Clause 18, wherein the nanoparticle-probe conjugates comprise one or more of thrombin, tissue plasminogen activator (TPA), streptokinase (SK), urokinase (uPA), heparin, protein S (PROS), or GAS 6.

Clause 20: A method for imaging and/or treating an irregularity in an anatomical lumen, the method comprising: providing an intraluminal device (such as in any one of Clauses 1-17) that includes a proximal end, a distal end, and a lumen extending therebetween through which a nanoparticle composition may be delivered, and a distal portion with an imaging device, a treatment device, or both associated therewith; advancing the intraluminal device within the anatomical lumen; delivering the nanoparticle composition to the anatomical lumen and allowing the nanoparticle composition to preferentially interact with an irregularity of the lumen; and performing one or both of activating the imaging device to image the lumen with images enhanced by the nanoparticles; or activating the treatment device to cause the nanoparticles to increase in motion.

Clause 21: The method of Clause 20, wherein the intraluminal device is a balloon device, the method further comprising inflating a balloon of the balloon device in the anatomical lumen so as to contact the balloon with the irregularity of the lumen, and activating the treatment device so as to transmit energy through the balloon and to the irregularity of the lumen to thereby cause the nanoparticles to increase in motion.

Clause 22: The method of Clause 21, wherein the energy transmitted through the balloon is ultrasound energy.

Clause 23: The method of any one of Clauses 20-22, wherein the nanoparticle composition comprises particle-probe conjugates configured to preferentially interact with the irregularity of the lumen.

Clause 24: The method of any one of Clauses 20-23, wherein the lumen is a blood vessel and the irregularity is a plaque.

Clause 25: The method of any one of Clauses 20-24, further comprising expanding one or more occluders within the target lumen to limit passage of the nanoparticle composition beyond the vicinity of the distal end of the intraluminal device.

Clause 26: An intraluminal system configured to be at least partially positioned within an anatomical lumen of a subject, the intraluminal system comprising: an intraluminal device having a length extending between a proximal end and a distal end, the intraluminal device defining a lumen along the length thereof through which a nanoparticle composition may be delivered to a targeted location within the anatomical lumen, the intraluminal device including a distal portion with a treatment device associated therewith, the treatment device configured to deliver energy to nanoparticles of the nanoparticle composition delivered through the lumen to activate the nanoparticles.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, or less than 0.1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intraluminal system configured to be at least partially positioned within an anatomical lumen of a subject, the intraluminal system comprising:
an intraluminal device having a length extending between a proximal end and a distal end, the intraluminal device defining a lumen along the length thereof through which a nanoparticle composition may be delivered, the intraluminal device including a distal portion with an imaging device associated therewith, the imaging device configured to image an intraluminal space and to generate nanoparticle-enhanced image data; and
an external device operatively coupled to the imaging device, the external device configured to receive the image data from the imaging device.

2. The intraluminal system of claim 1, further comprising a transmitter and a receiver, the transmitter coupled to the intraluminal device and the receiver coupled to the external device.

3. The intraluminal system of claim 2, further comprising a housing associated with a proximal portion of the intraluminal device, the transmitter being integrated with the housing.

4. The intraluminal system of claim 3, further comprising one or more power and/or data wires connecting the imaging device at the distal portion of the intraluminal device to the transmitter at the proximal portion of the intraluminal device.

5. The intraluminal system of claim 1, wherein the external device operates to display the image data received from the imaging device on a display screen of the external device to thereby display nanoparticle-enhanced images of the intraluminal space.

6. The intraluminal system of claim 1, wherein the lumen of the intraluminal device includes a lumen wall defining the lumen of the intraluminal device, the imaging device being integrated with the lumen wall.

7. The intraluminal system of claim 1, wherein the imaging device comprises an ultrasound transducer.

8. The intraluminal system of claim 7, wherein the ultrasound transducer is configured as a forward-looking ultrasound transducer.

9. The intraluminal system of claim 7, wherein the ultrasound transducer is configured as a side-looking ultrasound transducer.

10. The intraluminal system of claim 1, further comprising a treatment device associated with the distal portion of the intraluminal device, wherein the treatment device is configured to apply energy to the intraluminal space to activate nanoparticles disposed therein.

11. The intraluminal system of claim 10, further comprising an ultrasound transducer or an array of ultrasound transducers configured to function as both the imaging device and the treatment device.

12. The intraluminal system of claim 11, wherein the ultrasound transducer or the array of ultrasound transducers operate at a first frequency when functioning as the imaging device and operate at a second, different frequency when functioning as the treatment device.

13. The intraluminal system of claim 1, further comprising one or more occluders configured to limit passage of the nanoparticle composition beyond an area near the distal end of the intraluminal device.

14. The intraluminal system of claim 1, wherein the intraluminal device is a catheter.

15. The intraluminal system of claim 1, wherein the intraluminal device is a guidewire.

16. A kit for imaging and/or treating an irregularity in an anatomical lumen, the kit comprising:
an intraluminal device that includes
a proximal end, a distal end, and a lumen extending therebetween through which a nanoparticle composition may be delivered, and
a distal portion with an imaging device, a treatment device, or both associated therewith; and
a nanoparticle composition for passing through the lumen of the intraluminal device, the nanoparticle composition comprising nanoparticle-probe conjugates, the nanoparticle-probe conjugates comprising one or more probe molecules coupled to the nanoparticles.

17. The kit of claim 16, wherein the nanoparticle-probe conjugates comprise one or more of thrombin, tissue plasminogen activator (TPA), streptokinase (SK), urokinase (uPA), heparin, protein S (PROS), or GAS 6.

18. A method for imaging and/or treating an irregularity in an anatomical lumen, the method comprising:
providing an intraluminal device that includes
a proximal end, a distal end, and a lumen extending therebetween through which a nanoparticle composition may be delivered, and
a distal portion with an imaging device, a treatment device, or both associated therewith;
advancing the intraluminal device within the anatomical lumen;
delivering the nanoparticle composition to the anatomical lumen and allowing the nanoparticle composition to preferentially interact with an irregularity of the lumen; and performing one or both of
activating the imaging device to image the lumen with images enhanced by the nanoparticles;
activating the treatment device to cause the nanoparticles to increase in motion.

19. The method of claim 18, wherein the nanoparticle composition comprises particle-probe conjugates configured to preferentially interact with the irregularity of the lumen.

20. The method of claim 18, wherein the lumen is a blood vessel and the irregularity is a plaque.

21. The method of claim 18, further comprising expanding one or more occluders within the target lumen to limit passage of the nanoparticle composition beyond the vicinity of the distal end of the intraluminal device.
